(19) United States
(12) Patent Application Publication
(10) Pub. No.: US 2023/0172463 A1
(43) Pub. Date: Jun. 8, 2023

(54) NON-INVASIVE DEVICE FOR CONTINUOUS HEMODYNAMIC MONITORING UTILIZING A MICRO-LASER

(71) Applicant: Edwards Lifesciences Corporation, Irvine, CA (US)

(72) Inventors: Muhammad Mujeeb-U-Rahman, Irvine, CA (US); Jacobus Jozef Gerardus Maria Settels, De Hoef (NL)

(21) Appl. No.: 18/161,429

(22) Filed: Jan. 30, 2023

Related U.S. Application Data
(63) Continuation of application No. PCT/US21/17270, filed on Feb. 9, 2021.
(60) Provisional application No. 63/060,436, filed on Aug. 3, 2020.

(51) Int. Cl. A61B 5/022; A61B 5/00

(52) U.S. Cl. CPC A61B 5/02241; A61B 5/0059

(57) ABSTRACT

Disclosed is a finger cuff connectable to a patient's finger to be used in measuring the patient's blood pressure utilizing the volume clamp method. The finger cuff may comprise: a finger cavity to receive the patient's finger; a micro-laser and a detector to measure a pleth signal; a bladder mountable within the finger cavity, wherein the patient's finger received in the finger cavity abuts against the bladder; and a processor. The processor may be configured to control pressure applied by the bladder to the patient's finger based upon measuring the pleth signal received from the detector and the micro-laser to keep the pleth signal approximately constant to replicate the patient's blood pressure to implement the volume clamp method and to measure the patient's blood pressure.

### CROSS-REFERENCE TO RELATED APPLICATIONS

This application claims priority to PCT/US2021/017270 filed on Feb. 9, 2021, which claims priority to U.S. Provisional Patent Application No. 63/060,436, filed on Aug. 3, 2020, the entireties of each of which are hereby incorporated by reference.

### BACKGROUND

### Field

Embodiments of the invention relate generally to a non-invasive device for hemodynamic monitoring. More particularly, embodiments relate to a finger cuff to be used in measuring the patient's blood pressure utilizing the volume clamp method, in which, the finger cuff utilizes a micro-laser.

### Relevant Background

Non-invasive volume clamp cuffs are a revolutionary technology for continuous hemodynamic monitoring without the need of invasive procedures. These devices use an optical transmitter to generate a signal at a desired wavelength that passes through a biological tissue (e.g., a finger) and a detector that detects the amount of light after passing through this tissue. The device also houses an inflatable bladder to compress the tissue (e.g., the finger) with a known pressure to determine the effect of such compression on the signal response. When used together with a proper understanding of tissue perfusion, such a system can be used to monitor hemodynamic parameters (e.g., blood pressure) non-invasively.

As an example, volume clamping is a technique for non-invasively measuring blood pressure in which pressure is applied to a patient's finger in such a manner that arterial pressure may be balanced by a time varying pressure to maintain a constant arterial volume. In a properly fitted and calibrated system, the applied time varying pressure is approximately equal to the arterial blood pressure in the finger. The applied time varying pressure may be measured to provide a reading of the patient's blood pressure. This may be accomplished by a finger cuff volume clamp device that is arranged or wrapped around a finger of a patient. The finger cuff may include an optical source, an optical detector, and an inflatable bladder. The light may be sent from the optical source through the finger in which a finger artery is present. The optical detector picks up the light and the amount of light registered by the detector may be inversely proportional to the artery diameter and indicative of the pressure in the artery. In the finger cuff implementation, by inflating the bladder in the finger cuff, a pressure is exerted on the finger and finger artery. If the pressure is high enough, it will compress the artery causing the artery diameter to become smaller and the amount of light registered by the detector will increase. The amount of pressure necessary in the inflatable bladder to compress the artery is dependent on the blood pressure. By controlling the pressure of the inflatable bladder such that the diameter of the finger artery is kept constant, the blood pressure may be monitored in very precise detail as the pressure in the inflatable bladder is directly linked to the patient's blood pressure. In a typical present-day finger cuff implementation, a volume clamp system is used with the finger cuff. The volume clamp system typically includes a pressure generating system and a regulating system that includes: a pump, a valve, a controller, and a pressure sensor in a closed loop feedback system that is used in the measurement of the arterial volume. To accurately measure blood pressure, the feedback loop provides sufficient pressure generating and releasing capabilities to match the pressure oscillations of the patient's blood pressure.

As has been described, a finger cuff, as part of a volume clamp system, is used to measure the patient's blood pressure at their finger (e.g., the finger cuff being wrapped around the patient's finger). As an example, a controller may control the pneumatic pressure applied to the finger cuff by the pump as well as many other functions. In one example, the pneumatic pressure applied by the pump to the bladder of the finger cuff to replicate the patient's blood pressure may be calculated by the controller and may be based upon measuring the plethysmograph/plethysmogram (pleth) signal received from the optical source and detector pair of the finger cuff (e.g., to keep the pleth signal constant) and further the controller may measure the patient's blood pressure by monitoring the pressure of the bladder from a pressure sensor.

Current designs of volume clamp finger cuff devices use a single Light Emitting Diode (LED) and a photodetector (PD). In current finger cuffs, an LED transmits a signal spread from the LED to the PD through the patient's finger placed within the finger cuff. In particular, the LED output inherently has a spread that increases with distance and hence only part of the signal from the LED reaches the PD surface. This implementation results in some limitations in system performance. One example limitation is the decrease in the optical signal when used for neonates and infants as the smaller tissue length absorbs less light compared to an adult tissue. The other limitation is that the LED and the PD are placed in the middle of the bladder for better alignment of the transmitted and received signal. However, this means that a tradeoff must be made between the size of optical devices and the proper inflation and deflation of the bladder. Therefore, it would be beneficial to have an improved finger cuff design that eliminates the limitations of current designs resulting in better performance suitable for a wider variety of patients under wider use cases.

### BRIEF DESCRIPTION OF THE DRAWINGS

FIG. 1 is a diagram of an example of a system with a finger cuff, according to one optional example.

FIG. 2 is a block diagram of a finger cuff system to measure the blood pressure of a patient, according to one optional example.

FIG. 3A is a diagram of a finger cuff utilizing a micro-laser embedded within the finger cuff, according to one optional example.

FIG. 3B is a diagram of a finger cuff utilizing the micro-laser embedded within the finger cuff, with a patient's finger placed in the finger cavity, according to one optional example.

FIG. 4 is a diagram of a finger cuff utilizing a micro-laser and a detector positioned on the exterior of the finger cuff, with a patient's finger placed in the finger cavity, according to one optional example

FIG. 5 is a diagram of a finger cuff utilizing a micro-laser, a detector, and a reflector, with a patient's finger placed in the finger cavity, according to one optional example

FIG. 6 is a diagram of a finger cuff utilizing multiple micro-lasers and multiple detectors positioned on the exterior of the finger cuff, with a patient's finger placed in the finger cavity, according to one optional example

### SUMMARY

In one embodiment, a finger cuff connectable to a patient's finger to be used in measuring the patient's blood pressure by a blood pressure measurement system utilizing the volume clamp method is disclosed. The finger cuff may comprise: a finger cavity to receive the patient's finger; a micro-laser and a detector to measure a plethysmograph (pleth) signal; a bladder mountable within the finger cavity, wherein the patient's finger received in the finger cavity abuts against the bladder; and a processor. The processor may be configured to control pressure applied by the bladder to the patient's finger based upon measuring the pleth signal received from the detector and the micro-laser to keep the pleth signal approximately constant to replicate the patient's blood pressure to implement the volume clamp method and to measure the patient's blood pressure.

In one optional example, the micro-laser and the detector are embedded on the interior of the finger cavity of the finger cuff to be adjacent and in close proximity to the patient's finger. In one optional example, the micro-laser and the detector are positioned on the exterior of the finger cavity of the finger cuff. In one optional example, a reflector may be used, wherein, the micro-laser emits a signal through the patient's finger, and the signal is reflected by the reflector back through the patient's finger to the detector. This is used to increase the path length of the signal through the finger e.g. in case of children with very small fingers. In one optional example, a plurality of pairs of micro-lasers and detectors may be used to generate and receive a plurality of signals through the patient's finger. In one optional example, the micro-laser may be a vertical cavity surface emitting laser (VCSEL). It should be appreciated that the optional examples may be utilized independently from one another or in combination with one another.

In one embodiment, a system to measure a patient's blood pressure is disclosed. The system may include a finger cuff connectable to a patient's finger to be used in measuring the patient's blood pressure by a blood pressure measurement system utilizing the volume clamp method. The finger cuff may comprise: a finger cavity to receive the patient's finger; a micro-laser and a detector to measure a pleth signal; a bladder mountable within the finger cavity, wherein the patient's finger received in the finger cavity abuts against the bladder; and a processor. The processor may be configured to control pressure applied by the bladder to the patient's finger based upon measuring the pleth signal received from the detector and the micro-laser to keep the pleth signal approximately constant to replicate the patient's blood pressure to implement the volume clamp method and to measure the patient's blood pressure.

In one optional example, the micro-laser and the detector are embedded on the interior of the finger cavity of the finger cuff to be adjacent and in close proximity to the patient's finger. In one optional example, the micro-laser and the detector are positioned on the exterior of the finger cavity of the finger cuff. In one optional example, a reflector may be used, wherein, the micro-laser emits a signal through the patient's finger, and the signal is reflected by the reflector back through the patient's finger to the detector. In one optional example, a plurality of pairs of micro-lasers and detectors may be used to generate and receive a plurality of signals through the patient's finger. In one optional example, the micro-laser may be a vertical cavity surface emitting laser (VCSEL). It should be appreciated that the optional examples may be utilized independently from one another or in combination with one another.

In one embodiment, a method to measure a patient's blood pressure by a finger cuff connectable to a patient's finger with a blood pressure measurement system utilizing the volume clamp method is disclosed. The method may comprise: attaching the finger cuff to the patient's finger, wherein the patient's finger received in a finger cavity of the finger cuff abuts against a bladder mounted within the finger cavity; and controlling pressure applied by the bladder to the patient's finger based upon measuring a pleth signal received from a micro-laser and a detector of the finger cuff to keep the pleth signal approximately constant to replicate the patient's blood pressure to implement the volume clamp method and to measure the patient's blood pressure.

In one optional example, the micro-laser and the detector are embedded on the interior of the finger cavity of the finger cuff to be adjacent and in close proximity to the patient's finger. In one optional example, the micro-laser and the detector are positioned on the exterior of the finger cavity of the finger cuff. In one optional example, a reflector may be used, wherein, the micro-laser emits a signal through the patient's finger, and the signal is reflected by the reflector back through the patient's finger to the detector. In one optional example, a plurality of pairs of micro-lasers and detectors may be used to generate and receive a plurality of signals through the patient's finger. In one optional example, the micro-laser may be a vertical cavity surface emitting laser (VCSEL). It should be appreciated that the optional examples may be utilized independently from one another or in combination with one another.

### DETAILED DESCRIPTION

Embodiments of the invention generally relate to a non-invasive volume clamp device that eliminates the limitations of previously described designs that utilize LEDs as set forth in the background. Embodiments provided herein result in better performance, suitable for a wider variety of patients under wider use cases. In particular, embodiments of the non-invasive volume clamp finger cuff is based upon a sensing platform that utilizes one or more micro-lasers instead of a single LED. The use of a micro-laser offers unique benefits in terms of optical coherence and a more focused and less scattered signal. This means signal reflection can be used more to increase path length through the tissue to improve the overall signal. As an example, this may be useful for a neonate application.

As will be described in more detail hereafter, one example of a finger cuff may relate to a finger cuff that is connectable to a patient's finger to be used in measuring the patient's blood pressure by a blood pressure measurement system utilizing the volume clamp method. The finger cuff may comprise: a finger cavity to receive the patient's finger; a micro-laser and a detector to measure a pleth signal; a bladder mountable within the finger cavity, wherein the patient's finger received in the finger cavity abuts against the bladder; and a processor. The processor may be configured to control pressure applied by the bladder to the patient's finger based upon measuring the pleth signal received from the detector and the micro-laser to keep the pleth signal approximately constant to replicate the patient's blood pressure to implement the volume clamp method and to measure the patient's blood pressure.

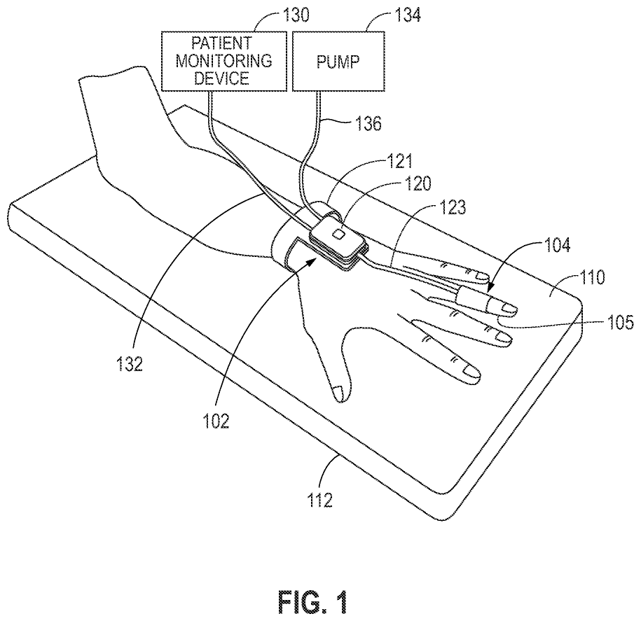

As an optional example, with reference to FIG. 1, which illustrates an example of a blood pressure measurement system, a blood pressure measurement system 102 that includes a finger cuff 104 that may be attached to a patient's finger 105 and a blood pressure measurement controller 120, which may be attached to the patient's body (e.g., a patient's wrist or hand), is shown. The blood pressure measurement system 102 may further be connected to a patient monitoring device 130, and, in some examples, a pump 134. The finger cuff 104 may be formed from a flexible material that is wrapped around the patient's finger such that the patient's finger is received in a finger cavity of the finger cuff. Further, the finger cuff 104 may include a bladder (not shown) and an optical source and optical sensor pair (not shown). In an optional example, a conventional pressure generating and regulating system may be utilized, in which, a pump 134 is located remotely from the body of the patient. In this example, the blood pressure measurement controller 120 receives pneumatic pressure from remote pump 134 through tube 136 and passes on the pneumatic pressure through tube 123 to the bladder of finger cuff 104. Blood pressure measurement device controller 120 may also control the pneumatic pressure (e.g., utilizing a controllable valve) applied to the finger cuff 104 as well as other functions. In this example, the pneumatic pressure applied by the pump 134 to the bladder of finger cuff 104 to replicate the patient's blood pressure based upon measuring the pleth signal received from the optical source and optical sensor pair of the finger cuff 104 (e.g., to keep the pleth signal constant) and measuring the patient's blood pressure by monitoring the pressure of the bladder may be controlled by the blood pressure measurement controller 120 and/or a remote computing device and/or the pump 134 and/or the patient monitoring device 130 to implement the volume clamping method. In some examples, a blood pressure measurement controller 120 is not used at all and there is simply a connection from tube 136 from a remote pump 134 including a remote pressure regulatory system to finger cuff 104, and all processing for the pressure generating and regulatory system, data processing, and display is performed by a remote computing device. Also, in some optional examples, the blood pressure measurement system 102 may include a pressure measurement controller 120 that includes: a small internal pump, a small internal valve, a pressure sensor, and control circuitry, to implement the volume clamping method. It should be appreciated that the finger cuff 104 may be connected to a blood pressure measurement controller described herein, or a pressure generating and regulating system of any other kind, such as a pressure generating and regulating system that is located remotely from the body of the patient. Any kind of pressure generating and regulating system can be used, including but not limited to the blood pressure measurement controller, and may be described simply as a pressure generating and regulating system that may be used with a finger cuff 104 including an optical source and detector pair and a bladder to implement the volume clamping method.

Continuing with this example, as shown in FIG. 1, a patient's hand may be placed on the face 110 of an arm rest 112 for measuring a patient's blood pressure with the blood pressure measurement system 102. The blood pressure measurement controller 120 of the blood pressure measurement system 102 may be coupled to a bladder of the finger cuff 104 in order to provide pneumatic pressure to the bladder for use in blood pressure measurement. Blood pressure measurement controller 120 may be coupled to the patient monitoring device 130 through a power/data cable 132. Also, in one example, as previously described, in a remote implementation, blood pressure measurement controller 120 may be coupled to a remote pump 134 through tube 136 to receive pneumatic pressure for the bladder of the finger cuff 104. The patient monitoring device 130 may be any type of medical electronic device that may read, collect, process, display, etc., physiological readings/data of a patient including blood pressure, as well as any other suitable physiological patient readings. Accordingly, power/data cable 132 may transmit data to and from patient monitoring device 130 and also may provide power from the patient monitoring device 130 to the blood pressure measurement controller 120 and finger cuff 104. In an optional example, the patient monitoring device 130 may be in wireless communication with the blood pressure measurement controller 120 and finger cuff 104 without the use of a wire.

As can be seen in FIG. 1, in one example, the finger cuff 104 may be attached to a patient's finger and the blood pressure measurement controller 120 may be attached on the patient's hand or wrist with an attachment bracelet 121 that wraps around the patient's wrist or hand. The attachment bracelet 121 may be metal, plastic, Velcro, etc. It should be appreciated that this is just one example of attaching a blood pressure measurement controller 120 and that any suitable way of attaching a blood pressure measurement controller to a patient's body or in close proximity to a patient's body may be utilized and that, in some examples, a blood pressure measurement controller 120 may not be used at all. It should further be appreciated that the finger cuff 104 may be connected to a blood pressure measurement controller described herein, or a pressure generating and regulating system of any other kind, such as a pressure generating and regulating system that is located remotely from the body of the patient. Any kind of pressure generating and regulating system can be used, including but not limited to the blood pressure measurement controller, and may be described simply as a pressure generating and regulating system that may be used with a finger cuff 104 including optical source and optical sensor pair and a bladder to implement the volume clamping method. In one optional example, the blood pressure measurement controller 120 and finger cuff 104 are a wearable device that may be in wireless communication with the patient monitoring device 130, as previously described.

Embodiments of the invention generally relate to a non-invasive volume clamp device that eliminates the limitations of previously described designs that utilize LEDs as set forth in the background. Embodiments provided herein result in better performance, suitable for a wider variety of patients under wider use cases. In particular, embodiments of the non-invasive volume clamp finger cuff 104 is based upon a sensing platform that utilizes one or more micro-lasers instead of a single LED. The use of a micro-laser offers unique benefits in terms of optical coherence and a more focused and less scattered signal. This means signal reflection can be used more to increase path length through the tissue to improve the overall signal. As an example, this may be useful for a neonate application.

As has been described, one example of a finger cuff 104 may relate to a finger cuff that is connectable to a patient's finger to be used in measuring the patient's blood pressure by a blood pressure measurement system utilizing the volume clamp method. The finger cuff may comprise: a finger cavity to receive the patient's finger; a micro-laser and a detector to measure a pleth signal; a bladder mountable within the finger cavity, wherein the patient's finger received in the finger cavity abuts against the bladder; and a processor. The processor may be configured to control pressure applied by the bladder to the patient's finger based upon measuring the pleth signal received from the detector and the micro-laser to keep the pleth signal approximately constant to replicate the patient's blood pressure to implement the volume clamp method and to measure the patient's blood pressure.

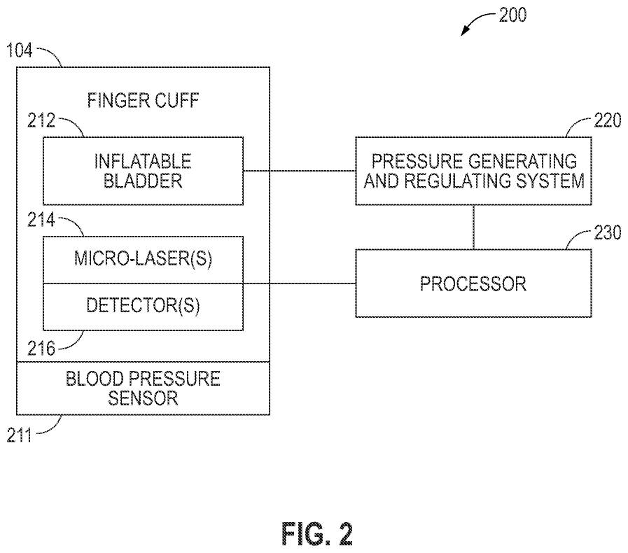

With additional reference to FIG. 2, FIG. 2 is a block diagram of a blood pressure measurement system 200 utilizing a finger cuff 104 to measure the blood pressure of a patient, according to one optional example. As an example, finger cuff 104 may be connectable to a patient's finger 105 to be used in measuring the patient's blood pressure by the blood pressure measurement system utilizing the volume clamp method. As has been described, finger cuff 104 may be wrapped around a patient's finger and may include a finger cavity to receive the patient's finger. Further, finger cuff 104 may include micro-laser 214 and a detector 216 to measure a pleth signal. It should be noted that a one micro-laser 214 and detector 216 may be used or, as will be described, multiple micro-lasers 214 and detectors 216 may be used. Moreover, finger cuff 104 may include an inflatable bladder 212 mountable within the finger cavity of the finger cuff, in which, the patient's finger 105 received in the finger cavity abuts against the inflatable bladder 212 such that the bladder 212 and the micro-laser and detector pair 214, 216 are used in measuring the patient's blood pressure information utilizing the volume clamp method. Additionally, processor 230 of the finger cuff 104 may be configured to control pressure applied by the bladder 212 to the patient's finger 105 by the pressure generating and regulating system 220 to replicate the patient's blood pressure based upon measuring the pleth signal by the micro-laser and detector pair 214, 216 (e.g., to keep the pleth signal approximately constant) to implement the volume clamp method.

The finger cuff 104 may be a finger cuff, as previously described, in which, the bladder 212 is an inflatable bladder that may be pneumatically connected to the pressure generating and regulating system 220. The micro-laser 214 may be used to illuminate the finger skin and light absorption or reflection may be detected with the detector 216. The pressure generating and regulating system 220 and processor 230 may generate, measure, and regulate pneumatic pressure that inflates or deflates the bladder 212, and may further comprise such elements as a pump, a valve, a pressure sensor, and/or other suitable elements, as previously described. In particular, pressure generating and regulating system 220 in cooperation with processor 230 may be configured to implement a volume clamp method with the finger cuff 104 by: applying pneumatic pressure to the bladder 212 of the finger cuff 104 to replicate the patient's blood pressure based upon measuring the pleth signal received from the micro-laser and detector pair 214, 216 of the finger cuff 104 (e.g., to keep the pleth signal approximately constant); and measuring the patient's blood pressure by monitoring the pressure of the bladder 212 based upon input from a blood pressure sensor 211, which should correspond to or be the same as patient's blood pressure, and may further command the display of the patient's blood pressure on the patient monitoring device 130 via wired or wireless communication. As previously described, in one optional example, the finger cuff 104, pressure generating and regulating system 220, and control circuitry 230 may be a wearable device that may be in wireless communication with the patient monitoring device 130.

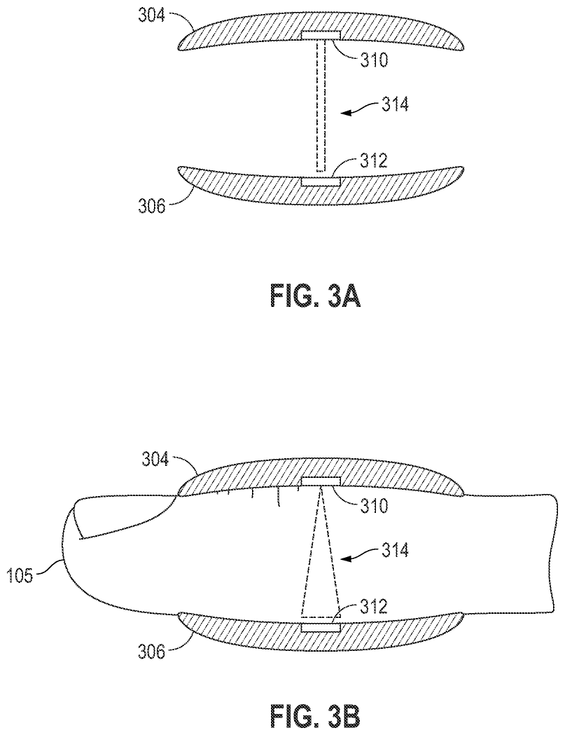

With additional reference to FIG. 3A, FIG. 3A is a diagram of a finger cuff utilizing a micro-laser embedded within the finger cuff, according to one optional example. As can be seen in FIG. 3A, the laser signal 314 from the micro-laser 310 embedded with the top 304 of the finger cuff to the detector 312 in the bottom 306 of the finger cuff has a narrow amount of spread (in comparison to a typical LED implementation), and hence almost all of the laser signal 314 reaches the detector 312 surface. With additional reference to FIG. 3B, FIG. 3B is a diagram of the finger cuff utilizing the micro-laser 310 embedded within the finger cuff, with a patient's finger placed in the finger cavity, according to one optional example. In FIG. 3B, an optional example of a finger cuff is shown that is connectable to a patient's finger 105 to be used in measuring the patient's blood pressure by a blood pressure measurement system utilizing the volume clamp method. The finger cuff may comprise: a finger cavity to receive the patient's finger 105 and a micro-laser 310 and a detector 312 to measure a pleth signal. In this optional example, the micro-laser 310 and the detector 312 are embedded on the interior of the finger cavity of the finger cuff to be adjacent and in close proximity to the patient's finger 105. A bladder 212 may be mounted within the finger cavity, wherein the patient's finger received in the finger cavity abuts against the bladder. As has been described, processor 230 may be configured to control pressure applied by the bladder to the patient's finger based upon measuring the pleth signal received from the detector 312 and the micro-laser 310 to keep the pleth signal approximately constant to replicate the patient's blood pressure to implement the volume clamp method and to measure the patient's blood pressure. As can be seen in FIG. 3B, by utilizing a micro-laser 310 embedded within the top 304 of the finger cuff, a very well defined spread of the laser signal 314 through the patient's finger 105 to the detector 312 embedded within the bottom 306 of the finger cuff is achieved for optimal collimation at the detector for more accurate pleth signal readings (as opposed to typical LED implementations).

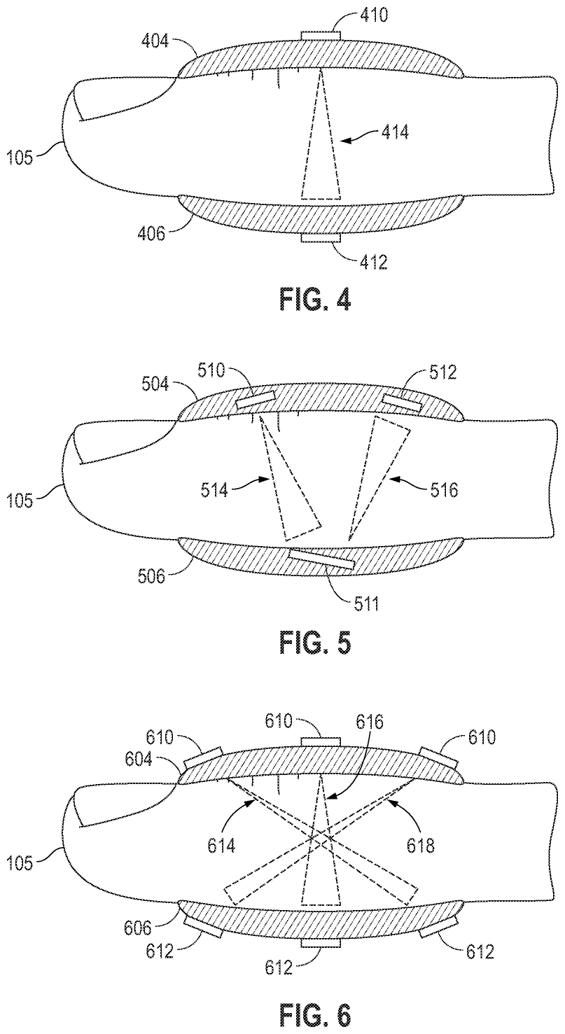

With additional reference to FIG. 4, FIG. 4 is a diagram of a finger cuff utilizing a micro-laser 410 and a detector 412 positioned on the exterior of the finger cuff, with a patient's finger placed in the finger cavity, according to one optional example. In this optional example, a finger cuff is shown that is connectable to a patient's finger 105 to be used in measuring the patient's blood pressure by a blood pressure measurement system utilizing the volume clamp method. The finger cuff may comprise: a finger cavity to receive the patient's finger 105 and a micro-laser 410 and a detector 412 to measure a pleth signal. In this optional example, the micro-laser 410 is positioned on the exterior of the top 404 of the finger cuff and the detector 412 is positioned on the exterior of the bottom of the finger cuff. As has been described, a bladder 212 may be mounted within the finger cavity, wherein the patient's finger received in the finger cavity abuts against the bladder. As has been described, processor 230 may be configured to control pressure applied by the bladder to the patient's finger based upon measuring the pleth signal received from the detector 412 and the micro-laser 410 to keep the pleth signal approximately constant to replicate the patient's blood pressure to implement the volume clamp method and to measure the patient's blood pressure. As can be seen in FIG. 4, by utilizing a micro-laser 410 positioned on the exterior of the top of 404 of the finger cuff, a very well defined spread of the laser signal 414 through the patient's finger 105 to the detector 412 positioned on the exterior of the bottom 406 of the finger cuff is achieved for optimal collimation at the detector for more accurate pleth signal readings (as opposed to typical LED implementations). This configuration of the micro-laser 410 and detector 412 to the exterior provides the benefit of allowing providing a maximum area of inflation by the bladder in the interior finger cavity of the finger cuff to thereby improve device performance while maintaining optimal detection of the laser signal 414 by the detector 412 from the micro-laser 410.

With additional reference to FIG. 5, FIG. 5 is a diagram of a finger cuff utilizing a micro-laser 510, a detector 512, and a reflector 511, with a patient's finger placed in the finger cavity, according to one optional example. In this optional example, a finger cuff is shown that is connectable to a patient's finger 105 to be used in measuring the patient's blood pressure by a blood pressure measurement system utilizing the volume clamp method. The finger cuff may comprise: a finger cavity to receive the patient's finger 105, micro-laser 510, a reflector 511, and a detector 512 to measure a pleth signal. In this optional example, a reflector 511 embedded in the bottom 506 of the finger cuff may be used, in which, the micro-laser 510 (in the top 504 of the finger cuff) emits a laser signal 514 through the patient's finger 105, and the laser signal 514 is reflected by the reflector 511 back through the patient's finger 105 to the detector 512 (in the top 504 of the finger cuff). As has been described, a bladder 212 may be mounted within the finger cavity, wherein the patient's finger received in the finger cavity abuts against the bladder. As has been described, processor 230 may be configured to control pressure applied by the bladder to the patient's finger based upon measuring the pleth signal received from the detector 512 and the micro-laser 510 to keep the pleth signal approximately constant to replicate the patient's blood pressure to implement the volume clamp method and to measure the patient's blood pressure. As can be seen in FIG. 5, by utilizing a micro-laser 510 positioned on the top of 504 of the finger cuff, the laser signal 514 that is reflected by reflector 511 as laser signal 516 back to detector 512 provides a laser signal with increased path length through the biological tissue of the finger 105 to increase sensitivity while maintaining a very well defined spread to maintain optimal detection of the laser signal 516 by the detector 512 for more accurate pleth signal readings (as opposed to typical LED implementations).

With additional reference to FIG. 6, FIG. 6 is a diagram of a finger cuff utilizing multiple micro-lasers 610 and multiple detectors 612 positioned on the exterior of the finger cuff, with a patient's finger placed in the finger cavity, according to one optional example. In this optional example, a finger cuff is shown that is connectable to a patient's finger 105 to be used in measuring the patient's blood pressure by a blood pressure measurement system utilizing the volume clamp method. The finger cuff may comprise: a finger cavity to receive the patient's finger 105 and multiple micro-lasers 610 and multiple detectors 612 to measure a pleth signal. In this optional example, a plurality of pairs of micro-lasers 610 and detectors 612 may be used to generate and receive a plurality of laser signals 614, 616, 618 through the patient's finger 105. In this example, the micro-lasers 610 and detectors 612 of the plurality of pairs of micro-lasers 610 and detectors 612, may be positioned on the exterior of the top 604 and bottom 606 of the finger cuff, respectively. Further, as has been described, a bladder 212 may be mounted within the finger cavity, wherein the patient's finger received in the finger cavity abuts against the bladder. As has been described, processor 230 may be configured to control pressure applied by the bladder to the patient's finger based upon measuring the pleth signal received from the detectors 612 and micro-lasers 610 to keep the pleth signal approximately constant to replicate the patient's blood pressure to implement the volume clamp method and to measure the patient's blood pressure. As can be seen in FIG. 6, by utilizing micro-lasers 610 positioned on the exterior of the top of 604 of the finger cuff, a very well defined spread of the laser signal 614, 616, 618 through the patient's finger 105 to the detectors 612 positioned on the exterior of the bottom 606 of the finger cuff is achieved for optimal collimation at the detectors for more accurate pleth signal readings (as opposed to typical LED implementations). Further, this configuration of micro-lasers 610 and detectors 612 to the exterior provides the benefit of allowing providing a maximum area of inflation by the bladder in the interior finger cavity of the finger cuff to thereby improve device performance while maintaining optimal detection of the laser signals 614, 616, 618 by the detectors 612 from the micro-lasers 610. Moreover, the use of multiple micro-lasers 610 and multiple detectors 612 increases the amount of received laser signals 614, 616, 618, which, in turn, improves system sensitivity and accuracy.

In one optional example, the micro-lasers previously described may be vertical cavity surface emitting lasers (VCSELs). Such devices can provide an efficiency of 70%, a narrow beam angle (20°), with a perfect Gaussian profile, and narrow spectral width. Further, these devices are available in a variety of wavelengths ranging from red to near infrared/infrared wavelengths. In comparison, LEDs often have an efficiency of less than 50% (e.g., 20% typically), have much wider beams (e.g., viewing angles greater than 100°), and have less thermal stability and Non-Gaussian beam. As an example, during a measurement, a VCSEL with the same drive current can provide more than 60 times the on-axis power, when compared with an LED. Therefore, a VCSEL can provide significant improvements in system design, as compared to an LED. Additionally, the use of a VCSEL can reduce the complexity of system design by enabling lower-power drive electronics and a less involved detection circuit. Moreover, a single VCSEL or a combination of VCSELs, can be used to enable continuous operation, which can provide a more continuous waveform with richer information than that observed with a combination of LEDs requiring aggressive duty cycling for thermal management.

In addition, with the use of micro-lasers (e.g., VCSELs), improvements can be made to system size, weight, and cost by using smaller and more integrated electronics than prior LED implementations. The previously described example implementations significantly improve the performance of the volume clamp system. These implementations increase the efficiency of the bladder by providing more area to it, as the micro-lasers, can be placed outside of the finger cuff away from the bladder, as opposed to the inside the cuff, as is done with current LED-based designs. Moreover, these previously described implementations improve signal absorption for a wider patient population by increasing signal pass length through the tissue. This may be achieved by using micro-lasers at edges of the finger cuff, as well as, by having multiple passes through the biological tissue owing to the tightly collimated laser beam, which spreads lesser than an LED signal.

It should be appreciated that a VCSEL is just one type of micro-laser that may be utilized. It should be appreciated that there are wide variety of other types of lasers or micro-lasers than may be used and that provide similar functionality. It should be appreciated that the previously described implementations rely on transmission of the signal in the tissue and transmission that carries the information about tissue properties. However, in other implementations, the micro-laser and the detector can be used on the same side to make a device that works in reflection mode. Also, in some implementations, multiple excitation sources (e.g., multiple VCSELs) may be used in the system, such that more advanced signal processing can be used to extract more information from the system.

It should be appreciated that the various previously described optional example implementations may be utilized independently from one another or in combination with one another. For example, the implementation of FIGS. 3A and 3B including a single embedded micro-laser and detector may be used independently from the other implementations of FIGS. 4-6 or in combination with one or more of them, in a suitable configuration. Similarly, the implementation of FIG. 4 including a single exterior micro-laser and detector may be used independently from the other implementations of FIGS. 3A-3B, 5, and 6 or in combination with one or more of them, in a suitable configuration. Likewise, the implementation of FIG. 5 including a micro-laser, a reflector, and a detector may be used independently from the other implementations of FIGS. 3A-3B, 4, and 6 or in combination with one or more of them, in a suitable configuration. Similarly, the implementation of FIG. 6 including multiple pairs of micro-lasers and detectors may be used independently from the other implementations of FIGS. 3A-3B, 4, and 5 or in combination with one or more of them, in a suitable configuration. Accordingly, it should be appreciated that a wide variety of the previously described optional examples may be utilized independently from one another or in combination with one or more of them, in a suitable configuration.

It should be appreciated that FIG. 2 illustrates a non-limiting example of a processor 230 based implementation of the finger cuff system. As an example, the finger cuff system may comprise a processor, a memory, and an input/output connected with a bus. Under the control of the processor, data may be received from an external source through the input/output interface and stored in the memory, and/or may be transmitted from the memory to an external destination through the input/output interface. The processor may process, add, remove, change, or otherwise manipulate data stored in the memory. Further, code may be stored in the memory. The code, when executed by the processor, may cause the processor to perform operations relating to data manipulation and/or transmission and/or any other possible operations.

It should be appreciated that aspects of the invention previously described may be implemented in conjunction with the execution of instructions by processors, circuitry, controllers, etc. As an example, a processor may operate under the control of a program, algorithm, routine, or the execution of instructions to execute methods or processes in accordance with embodiments of the invention previously described. For example, such a program may be implemented in firmware or software (e.g. stored in memory and/or other locations) and may be implemented by circuitry, processors, and/or other circuitry, these terms being utilized interchangeably. Further, it should be appreciated that the terms processor, microprocessor, circuitry, control circuitry, circuit board, controller, microcontroller, etc., refer to any type of logic or circuitry capable of executing logic, commands, instructions, software, firmware, functionality, etc., which may be utilized to execute embodiments of the invention.

The various illustrative blocks, processors, modules, and circuitry described in connection with the embodiments disclosed herein may be implemented or performed with a general purpose processor, a specialized processor, circuitry, a microcontroller, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor may be a microprocessor or any conventional processor, controller, microcontroller, circuitry, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module/firmware executed by a processor, or any combination thereof. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

The disclosure also includes the following clauses:

1. A finger cuff connectable to a patient's finger to be used in measuring the patient's blood pressure by a blood pressure measurement system utilizing the volume clamp method, the finger cuff comprising:

a finger cavity to receive the patient's finger;

a micro-laser and a detector to measure a pleth signal;

a bladder mountable within the finger cavity, wherein the patient's finger received in the finger cavity abuts against the bladder; and

a processor configured to control pressure applied by the bladder to the patient's finger based upon measuring the pleth signal received from the detector and the micro-laser to keep the pleth signal approximately constant to replicate the patient's blood pressure to implement the volume clamp method and to measure the patient's blood pressure.

2. The finger cuff of clause 1, wherein, the micro-laser and the detector are embedded on the interior of the finger cavity of the finger cuff to be adjacent and in close proximity to the patient's finger.
3. The finger cuff of clause 1, wherein, the micro-laser and the detector are positioned on the exterior of the finger cavity of the finger cuff.
4. The finger cuff of any of the clauses 1-2, further comprising a reflector, wherein, the micro-laser emits a signal through the patient's finger, the signal is reflected by the reflector back through the patient's finger to the detector.
5. The finger cuff of any of the clauses 1-4, further comprising a plurality of pairs of micro-lasers and detectors to generate and receive a plurality of signals through the patient's finger.
6. The finger cuff of any of the clauses 1-5, wherein the micro-laser is a vertical cavity surface emitting laser (VCSEL).
7. A system to measure a patient's blood pressure, the system comprising:

a finger cuff connectable to a patient's finger to be used in measuring the patient's blood pressure by a blood pressure measurement system utilizing the volume clamp method, the finger cuff comprising:

a finger cavity to receive the patient's finger;

a micro-laser and a detector to measure a pleth signal;

a bladder mountable within the finger cavity, wherein the patient's finger received in the finger cavity abuts against the bladder; and

a processor configured to control pressure applied by the bladder to the patient's finger based upon measuring the pleth signal received from the detector and the micro-laser to keep the pleth signal approximately constant to replicate the patient's blood pressure to implement the volume clamp method and to measure the patient's blood pressure.

8. The system of clause 7, wherein, the micro-laser and the detector are embedded on the interior of the finger cavity of the finger cuff to be adjacent and in close proximity to the patient's finger.
9. The system of clause 7, wherein, the micro-laser and the detector are positioned on the exterior of the finger cavity of the finger cuff.
10. The system of any of the clauses 7-9, further comprising a reflector, wherein, the micro-laser emits a signal through the patient's finger, the signal is reflected by the reflector back through the patient's finger to the detector.
11. The system of any of the clauses 7-10, further comprising a plurality of pairs of micro-lasers and detectors to generate and receive a plurality of signals through the patient's finger.
12. The system of any of the clauses 7-11, wherein the micro-laser is a vertical cavity surface emitting laser (VCSEL).
13. A method to measure a patient's blood pressure by a finger cuff connectable to a patient's finger with a blood pressure measurement system utilizing the volume clamp method, the method comprising:

attaching the finger cuff to the patient's finger, wherein the patient's finger received in a finger cavity of the finger cuff abuts against a bladder mounted within the finger cavity; and

controlling pressure applied by the bladder to the patient's finger based upon measuring a pleth signal received from a micro-laser and a detector of the finger cuff to keep the pleth signal approximately constant to replicate the patient's blood pressure to implement the volume clamp method and to measure the patient's blood pressure.

14. The method of clause 13, wherein, the micro-laser and the detector are embedded on the interior of the finger cavity of the finger cuff to be adjacent and in close proximity to the patient's finger.
15. The method of clause 13, wherein, the micro-laser and the detector are positioned on the exterior of the finger cavity of the finger cuff.
16. The method of any of the clauses 13-15, further comprising a reflector, wherein, the micro-laser emits a signal through the patient's finger, the signal is reflected by the reflector back through the patient's finger to the detector.
17. The method of any of the clauses 13-16, further comprising a plurality of pairs of micro-lasers and detectors to generate and receive a plurality of signals through the patient's finger.
18. The method of any of the clauses 13-17, wherein the micro-laser is a vertical cavity surface emitting laser (VCSEL).

What is claimed is:

1. A finger cuff connectable to a patient's finger to be used in measuring the patient's blood pressure by a blood pressure measurement system utilizing the volume clamp method, the finger cuff comprising:
a finger cavity to receive the patient's finger;
a micro-laser and a detector to measure a pleth signal;
a bladder mountable within the finger cavity, wherein the patient's finger received in the finger cavity abuts against the bladder; and
a processor configured to control pressure applied by the bladder to the patient's finger based upon measuring the pleth signal received from the detector and the micro-laser to keep the pleth signal approximately constant to replicate the patient's blood pressure to implement the volume clamp method and to measure the patient's blood pressure.

2. The finger cuff of claim 1, wherein, the micro-laser and the detector are embedded on the interior of the finger cavity of the finger cuff to be adjacent and in close proximity to the patient's finger.

3. The finger cuff of claim 1, wherein, the micro-laser and the detector are positioned on the exterior of the finger cavity of the finger cuff.

4. The finger cuff of claim 1, further comprising a reflector, wherein, the micro-laser emits a signal through the patient's finger, the signal is reflected by the reflector back through the patient's finger to the detector.

5. The finger cuff of claim 1, further comprising a plurality of pairs of micro-lasers and detectors to generate and receive a plurality of signals through the patient's finger.

6. The finger cuff of claim 1, wherein the micro-laser is a vertical cavity surface emitting laser (VCSEL).

7. A system to measure a patient's blood pressure, the system comprising:
a finger cuff connectable to a patient's finger to be used in measuring the patient's blood pressure by a blood pressure measurement system utilizing the volume clamp method, the finger cuff comprising:
a finger cavity to receive the patient's finger;
a micro-laser and a detector to measure a pleth signal; and
a bladder mountable within the finger cavity, wherein the patient's finger received in the finger cavity abuts against the bladder; and
a processor configured to control pressure applied by the bladder to the patient's finger based upon measuring the pleth signal received from the detector and the micro-laser to keep the pleth signal approximately constant to replicate the patient's blood pressure to implement the volume clamp method and to measure the patient's blood pressure.

8. The system of claim 7, wherein, the micro-laser and the detector are embedded on the interior of the finger cavity of the finger cuff to be adjacent and in close proximity to the patient's finger.

9. The system of claim 7, wherein, the micro-laser and the detector are positioned on the exterior of the finger cavity of the finger cuff.

10. The system of claim 9, further comprising a reflector, wherein, the micro-laser emits a signal through the patient's finger, the signal is reflected by the reflector back through the patient's finger to the detector.

11. The system of claim 10, further comprising a plurality of pairs of micro-lasers and detectors to generate and receive a plurality of signals through the patient's finger.

12. The system of claim 11, wherein the micro-laser is a vertical cavity surface emitting laser (VCSEL).

13. A method to measure a patient's blood pressure by a finger cuff connectable to a patient's finger with a blood pressure measurement system utilizing the volume clamp method, the method comprising:
attaching the finger cuff to the patient's finger, wherein the patient's finger received in a finger cavity of the finger cuff abuts against a bladder mounted within the finger cavity; and
controlling pressure applied by the bladder to the patient's finger based upon measuring a pleth signal received from a micro-laser and a detector of the finger cuff to keep the pleth signal approximately constant to replicate the patient's blood pressure to implement the volume clamp method and to measure the patient's blood pressure.

14. The method of claim 13, wherein, the micro-laser and the detector are embedded on the interior of the finger cavity of the finger cuff to be adjacent and in close proximity to the patient's finger.

15. The method of claim 13, wherein, the micro-laser and the detector are positioned on the exterior of the finger cavity of the finger cuff.

16. The method of claim 15, further comprising a reflector, wherein, the micro-laser emits a signal through the patient's finger, the signal is reflected by the reflector back through the patient's finger to the detector.

17. The method of claim 16, further comprising a plurality of pairs of micro-lasers and detectors to generate and receive a plurality of signals through the patient's finger.

18. The method of claim 17, wherein the micro-laser is a vertical cavity surface emitting laser (VCSEL).
